(19) United States
(12) Patent Application Publication
(10) Pub. No.: US 2024/0263250 A1
(43) Pub. Date: Aug. 8, 2024

(54) InDel MOLECULAR MARKER OF hisD GENE OF ULTRASONICALLY-MUTAGENIZED SALMONELLA TYPHIMURIUM AND USE THEREOF

(71) Applicant: JIANGSU UNIVERSITY, Zhenjiang (CN)

(72) Inventors: Haile MA, Zhenjiang (CN); Siyu RUAN, Zhenjiang (CN); Yunliang LI, Zhenjiang (CN); Yaoyong JI, Zhenjiang (CN); Anqi ZHOU, Zhenjiang (CN); Pengfei XIE, Zhenjiang (CN); Bobo ZHAO, Zhenjiang (CN); Xiaoshuang LIU, Zhenjiang (CN); Yaxuan XU, Zhenjiang (CN); Yanling WU, Zhenjiang (CN)

(73) Assignee: JIANGSU UNIVERSITY, Zhenjiang (CN)

(21) Appl. No.: 17/760,099

(22) PCT Filed: Dec. 20, 2021
(86) PCT No.: PCT/CN2021/139459

§ 371 Date: Mar. 22, 2024

(30) Foreign Application Priority Data
Aug. 9, 2021 (CN) 202110909368.X

(51) Int. Cl. C12Q 1/689

(52) U.S. Cl. CPC C12Q 1/689; C12Q 2600/156

(57) ABSTRACT

An insertion and deletion (InDel) molecular marker of a hisD gene of ultrasonically-mutagenized Salmonella typhimurium (S. typhimurium) and use thereof are provided. Through ultrasonic mutagenesis to S. typhimurium and sequencing, it is found that all InDel mutations occur in a core mutation region of the hisD gene. Therefore, the core mutation region is used as the InDel molecular marker to determine an insertion or a deletion of a gene reverse mutation, and the InDel molecular marker can also be used to analyze a relationship between a sequence of the hisD gene and a function of a protein encoded thereby.

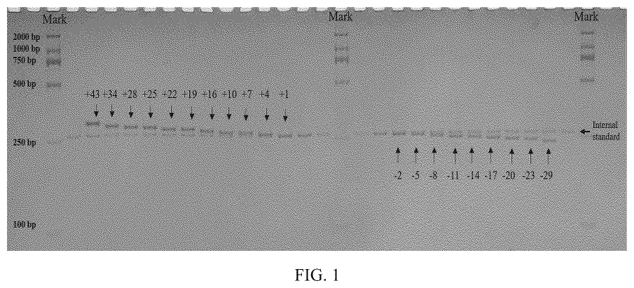

### CROSS REFERENCE TO THE RELATED APPLICATIONS

This application is the national phase entry of International Application No. PCT/CN2021/139459, filed on Dec. 20, 2021, which is based upon and claims priority to Chinese Patent Application No. 202110909368.X, filed on Aug. 9, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBNJIP074_Sequence_Listing.txt, created on 09/02/2022, and is 6,670 bytes in size.

### TECHNICAL FIELD

The present disclosure belongs to the technical fields of molecular biology and ultrasonic mutagenesis, and in particular relates to an insertion and deletion (InDel) molecular marker of a hisD gene of ultrasonically-mutagenized Salmonella typhimurium (S. typhimurium) and use thereof.

### BACKGROUND

Microorganisms play an increasingly important and irreplaceable role in solving human problems related food, energy, health, resources, and environmental protection, and also bring huge social and economic benefits to human beings. In order to acquire high-yield, low-consumption, and high-quality strains, mutation breeding is often conducted through physical, chemical, or biological factors. The physical mutagenesis has received widespread attention due to its advantages such as high safety, convenient operation, simple equipment, and high stability. For example, the biochemical degradation ability of activated sludge can be enhanced by changing a microbial composition of the activated sludge through ultraviolet (UV) radiation and optimizing a growth environment of target bacteria, and a mutant with more beneficial mutations that can be stably inherited by offspring can be obtained by ion implantation mutagenesis. A mutagenesis mechanism can be comprehensively and systematically studied through the femtosecond laser technology in combination with physical and biochemical reaction processes. Among them, the ultrasonic mutagenesis has received more and more attention due to its prominent directionality, strong penetrability, high safety, and easy control.

The research on ultrasonic mutagenesis mainly focuses on its biological effects, and there are few studies on its mutagenesis mechanism, which is not conducive to the further development of ultrasonic mutagenesis technology and ultrasonic equipment. The research on the mutagenesis mechanism relies on the accurate analysis of gene variation. The high-energy effects of physical fields such as ultrasonic fields can cause variations in DNA strands, and the variations are mainly divided into the following three types: 1. single nucleotide polymorphism (SNP); 2. InDel, which refers to an insertion or deletion of a small fragment sequence with a length usually of 50 bp or less at a specified position in genome; and 3. large structural variation (SV), which is common, including an insertion or deletion of a long fragment sequence with a length of 50 bp or more, a chromosome inversion, a sequence translocation within a chromosome or between chromosomes, a copy number variation, and some more complex variations. Type 2 and Type 3 variations are commonly referred to as genomic SV. In recent years, researchers have found that SVs have a greater impact on a genome than SNPs, SVs on a genome seem to be more suitable than SNPs for explaining the characteristics of species diversity, and some rare and identical SVs are often correlated with the occurrence of some diseases or even cause these diseases.

S. typhimurium is a non-adaptive or pantropic Salmonella species with a wide host spectrum, which is one of the main pathogenic bacteria causing acute gastroenteritis and is also a very important bacterial species in the development of microbial genetics. S. typhimurium is often modified into a live attenuated vaccine vector that is widely used in the research on immunization of various viruses, bacteria, parasites, and the like, which has achieved prominent results. The histidine-auxotrophic S. typhimurium reverse mutation assay (Ames test) can be used to detect the potential carcinogenicity of a chemical substance, which is a prominent method to observe a gene mutation and is widely used to screen the genetic toxicity and potential carcinogenicity of a chemical substance or a physical field. It is reported that the Ames test exhibits detection rates of 69.0% and 87.5% for carcinogens in rats and rhesus monkeys, respectively, and is also an important basis for a genetic toxicity screening database established by the quantitative structure-activity relationship (QSAR) of compounds.

Genes involved in histidine synthesis mainly include 9 genes such as hisA, hisB, hisC, hisD, hisE, hisF, hisG, hisH, and hisI. The mutation of these genes may cause the loss of histidine synthesis ability, and the normal gene functions of some mutated genes can be restored through reverse mutation, which is the molecular biology basis for the Ames test. Depending on the different types of gene reverse mutations, a variety of strains have been developed for the detection of mutation types. For point mutations or oligonucleotide strand (≤4 bp) mutations, four histidine-auxotrophic S. typhimurium strains (TA1535, TA1537/TA97/TA97a, TA98, and TA100) for detecting a base substitution or frameshift mutation at a guanine-cytosine (G-C) site in a histidine target gene and a histidine-auxotrophic S. typhimurium strain (TA102) for detecting a base substitution or frameshift mutation at an adenine-thymine (A-T) site in a histidine or tryptophan gene are developed, and for error-prone repair mutations caused by large-scale DNA damage, an SOS repair-based Umu test strain (TA1535) is developed. At present, there is no method for detecting an insertion or deletion of an oligonucleotide strand with a length of more than 4 bp in the S. typhimurium reverse mutation detection system, which is of great significance for the optimization and evaluation of physical field mutagenesis such as ultrasonic mutagenesis.

### SUMMARY

In view of the deficiencies in the prior art, the present disclosure provides an InDel molecular marker of a hisD gene of ultrasonically-mutagenized S. typhimurium and use thereof. In the present disclosure, through ultrasonic mutagenesis to S. typhimurium and sequencing of the hisD gene, it is found that all InDel mutations occur in a core mutation region of the hisD gene. Therefore, the core mutation region is used as the InDel molecular marker to determine an insertion or a deletion of a gene reverse mutation, and the InDel molecular marker can also be used to analyze a relationship between a sequence of the hisD gene and a function of a protein encoded thereby.

The present disclosure provides an InDel molecular marker of a hisD gene of ultrasonically-mutagenized S. typhimurium, where the InDel molecular marker is used to determine a type of an oligonucleotide insertion or deletion in an InDel core mutation region of the hisD gene, and the InDel molecular marker is a sequence from an 832^{nd} base to a 915^{th} base in the hisD gene of the S. typhimurium strain, and has a nucleotide sequence set forth in SEQ ID No: 4.

Further, the InDel molecular marker is amplified by the following specific molecular marker primers having the following sequences:

hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

The present disclosure also provides primers for detecting the InDel molecular marker described above, and nucleotide sequences of the primers are as follows:

hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

The present disclosure also provides use of the InDel molecular marker of the hisD gene of the ultrasonically-mutagenized S. typhimurium, which specifically includes using the molecular marker primers provided by the present disclosure to determine an insertion or a deletion of a gene reverse mutation.

The present disclosure also provides use of the InDel molecular marker of the hisD gene of the ultrasonically-mutagenized S. typhimurium described above in the direct analysis of a relationship between a sequence of the hisD gene and a function of a protein encoded by the sequence of the hisD gene.

The present disclosure also provides a method for detecting an oligonucleotide insertion or deletion in a hisD gene, including using the specific primers described above to amplify the InDel molecular marker to obtain an amplification product, where the nucleotide sequences of the primers are as follows:

hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

Further, in the method, with a DNA amplification product of a hisD gene of histidine-auxotrophic S. typhimurium as a control, polyacrylamide gel electrophoresis (PAGE) is performed, and if there is a difference in size between bands of the amplification product and the control, a reverse mutation of the oligonucleotide insertion or deletion occurs.

Further, in the method, the amplification product is sequenced to analyze a specific oligonucleotide sequence of the oligonucleotide insertion or deletion.

The present disclosure also provides use of the method described above in the detection of a reverse mutation in S. typhimurium mutagenized by a physical field selected from the group consisting of a pulsed electric field (PEF) and a femtosecond laser.

The present disclosure also provides a kit for detecting the InDel molecular marker described above, where the kit includes the primers for detecting the InDel molecular marker.

### Compared with the Prior Art, the Present Disclosure has the Following Beneficial Effects

In the present disclosure, based on the hisD gene sequencing data of 1,752 S. typhimurium revertants, a core mutation region of oligonucleotide insertions or deletions of the hisD gene is mapped, and then a molecular marker of the core mutation region is designed by narrowing the amplification range, and the PAGE method can be used to effectively determine a type of an insertion or deletion of an oligonucleotide strand of more than 4 bp in the hisD gene and accurately analyze a gene variation caused by an ultrasonic physical field, which is conducive to the further exploration of the mutagenesis mechanism.

Compared with the traditional sequencing method, the method for detecting an oligonucleotide insertion or deletion in the InDel core mutation region of the hisD gene provided by the present disclosure leads to a more intuitive result, and has a lower test cost and a higher application value.

In addition, the method for detecting an oligonucleotide insertion or deletion in the InDel core mutation region of the hisD gene provided by the present disclosure also has guiding significance for the detection of a reverse mutation of S. typhimurium mutagenized by a physical field such as PEF and femtosecond laser.

### BRIEF DESCRIPTION OF THE DRAWINGS

FIGURE is an electrophoretogram of all frameshift mutations (oligonucleotide insertions or deletions) in a core mutation region of hisD in TA98 revertants, where a PCR amplification product of hisD gene of a TA98 strain is used as an internal standard, and meanings of unique oligonucleotide insertion or deletion bands caused by ultrasound are shown in Table 1.

### DETAILED DESCRIPTION OF THE EMBODIMENTS

The present disclosure will be further described below in conjunction with the accompanying drawings and specific examples, but the protection scope of the present disclosure is not limited thereto.

Unless defined otherwise, all terms used in the present disclosure have the same meanings as commonly understood by one of ordinary skill in the art. In the following examples, various processes and methods that are not described in detail are conventional methods known in the art. The sources, trade names, and components needing to be listed of reagents are indicated when the reagents appear for the first time, and unless otherwise specified, the same reagents appearing thereafter are the same as those indicated for the first time. Unless otherwise specified, these reagents are all commercially available.

The equipment used for the ultrasonic treatment of the present disclosure is not limited. The TA98 strain of the present disclosure is purchased from MOLTOX™ Molecular Toxicology Inc., USA. The wild type (WT) S. typhimurium is a type of TA98 revertant, which has one more C base than the TA98 strain. The TA98 revertant is S. typhimurium that restores the histidine synthesis function.

### Example 1: Detection of the hisD Gene and Determination of the InDel Molecular Marker

### (1) Preparation of a Bottom Medium

16 mL of a phosphate stock solution, 20 mL of a 40% (w/v) glucose solution, and 0.6 mL of a 2 mM histidine-biotin solution were added in turn to 400 mL of a sterilized 1.5% (mass fraction) agar medium (80° ° C. to 90° C.), the resulting mixture was thoroughly mixed and used to prepare culture plates (75 mm plate) with 10 mL per plate, and after the mixture was condensed and solidified, the plates were invertedly incubated at 37° C. for 24 h.

(2) A TA98 bacterial suspension without ultrasonic treatment was adopted as a control group (TA98 was purchased from MOLTOX™ Molecular Toxicology Inc., USA), and a TA98 bacterial suspension undergoing ultrasonic treatment was adopted as an ultrasonic treatment group. A specific treatment method for the ultrasonic treatment group was as follows: 0.1 mL of a TA98 bacterial suspension undergoing ultrasonic treatment (a seed bacterial suspension was cultivated for 6 h and then subjected to ultrasonic treatment for 5 min at an ultrasonic power density of 20 W/mL and a frequency of 40 kHz) was taken and coated on a plate containing the bottom medium, and then cultivated in a constant-temperature incubator at 37ºC for 36 h to 72 h.

876 single colonies of the TA98 revertant resulting from ultrasonic mutagenesis in the ultrasonic treatment group and 876 single colonies of the TA98 revertant resulting from spontaneous mutation in the control group were separately picked (which was conducted through three parallel experiments, with a total of 1,752 colonies) and cultivated in the bottom liquid medium for 12 h at 37° C. and 100 r/min, and then the resulting bacterial suspension was collected for PCR amplification (pre-denaturation at 94ºC for 3 min, denaturation at 94° C. for 20 s, annealing at 55ºC for 30 s, and extension at 72ºC for 1.5 min, with 35 cycles in total; and extension at 72° C. for 10 min). A primer pair for the PCR amplification was as follows:

hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

(3) The PCR product was sent to Sangon Biotech (Shanghai) Co., Ltd. for sequencing, and a multiple sequence alignment tool MAFFT (https://www.ebi.ac.uk/Tools/msa/mafft/) was used to align sequencing results of the 1,752 TA98 revertants with the sequence of the TA98 strain.

The results showed that the frameshift mutations of the TA98 revertants exhibited polymorphic, and there were 127 insertion or deletion mutation types in the ultrasonic treatment group and the control group. These 127 insertion or deletion mutation types were concentrated in a core mutation region (a sequence from an 832^{nd} base to a 915^{th} base of the hisD gene, SEQ ID No: 4) of hisD gene (full length: 1,305 bp, SEQ ID No: 3). Therefore, the core mutation region was used as an InDel molecular marker, in which a unique oligonucleotide insertion or deletion mutation type appeared after the ultrasonic treatment, as shown in Table 1. The wild type S. typhimurium in Table 1 was a type of TA98 revertant, which had one more C base than the TA98 strain. Compared with the TA98 strain, base insertion sequences (>4 bp) of the TA98 revertants caused by ultrasound were all direct repeat sequences.

TABLE 1
DNA sequences of core mutation regions (No. 832 to No. 915) in hisD
gene of the TA98 revertants

| Type | DNA sequence |
|---|---|
| TA98 | GCCCGCAAGGTGGCGGAGGCGGTAGAACGTCAACTGGCGGAACTGC |
| | CGCGCGCGGACACCGCCGGCAGGCCCTGAGCGCCAGTC (SEQ ID No: |
| | 5) |
| Wild type | GCCCGCAAGGTGGCGGAGGCGGTAGAACGTCAACTGGCGGAACTGC |
| | CGCGCGCGGACACCGCCCGGCAGGCCCTGAGCGCCAGTC (SEQ ID |
| | No: 6) |
| +43 bp | GCCCGCAAGGTGGCGGAGGCGGTAGA(ACGTCAACTGGCGGAACTGC |
| | CGCGCGCGGACACCGCCGGCAGG)₂CCCTGAGCGCCAGTC (SEQ ID |
| | No: 7) |
| +34 bp | (GCCCGCAAGGTGGCGGAGGCGGTAGAACGTCAAC)₂TGGCGGAACTG |
| | CCGCGCGCGGACACCGCCGGCAGGCCCTGAGCGCCAGTC (SEQ ID |
| | No: 8) |
| +28 bp | GCCCGCAAGGTGGCGGAGGCGGTAGAACGTCAA(CTGGCGGAACTGC |
| | CGCGCGCGGACACCG)₂CCGGCAGGCCCTGAGCGCCAGTC (SEQ ID |
| | No: 9) |
| +25 bp | GCCCGCAAGGTGGCGGAGGCGGTAGAACGTCAAC(TGGCGGAACTGC |
| | CGCGCGCGGACAC)₂CGCCGGCAGGCCCTGAGCGCCAGTC (SEQ ID |
| | No: 10) |
| +22 bp | GCCCGCAAGGTGGCGGAGGCGGTAGAACGTCAACTGGCGGA(ACTGC |
| | CGCGCGCGGACACCGCC)₂GGCAGGCCCTGAGCGCCAGTC (SEQ ID |
| | No: 11) |
| +19 bp | GCCCGCAAGGTGGCGGAGGCGGTAGAACGTC(AACTGGCGGAACTGC |
| | CGCG)₂CGCGGACACCGCCGGCAGGCCCTGAGCGCCAGTC (SEQ ID |
| | No: 12) |
| +16 bp | GCCCGCAAGGTGGCGGAGGCGGTAGAACGTCAACTGGCGGAACTGC |
| | CG(CGCGCGGACACCGCCG)₂GCAGGCCCTGAGCGCCAGTC (SEQ ID |
| | No: 13) |
| +10 bp | GCCCGCAAGGTGGCGGAGGCGGTAGAACGTCA(ACTGGCGGAA)₂CTG |
| | CCGCGCGCGGACACCGCCGGCAGGCCCTGAGCGCCAGTC (SEQ ID |
| | No: 14) |
| +10 bp | GCCCGCAAGGTGGCGGAGGCGGTAGAACGTCAA(CTGGCGGAAC)₂TG |
| | CCGCGCGCGGACACCGCCGGCAGGCCCTGAGCGCCAGTC (SEQ ID |
| | No: 15) |
| +10 bp | GCCCGCAAGGTGGCGGAGGCGGTAGAACGTCAACTGGCGGAACTGC |
| | CGCGCGCGGACACCGCC(GGCAGGCCCT)₂GAGCGCCAGTC (SEQ ID |
| | No: 16) |
| +7 bp | GCCCGCAAGGTGGCGGAGGCGGTAGAA(CGTCAAC)₂TGGCGGAACTG |
| | CCGCGCGCGGACACCGCCGGCAGGCCCTGAGCGCCAGTC (SEQ ID |
| | No: 17) |
| +7 bp | GCCCGCAAGGTGGCGGAGGCGGTAGAACGTCAACTG(GCGGAAC)₂TG |
| | CCGCGCGCGGACACCGCCGGCAGGCCCTGAGCGCCAGTC (SEQ ID |
| | No: 18) |
| −29 bp | GCCCGCAAGGTGGCGGAGGCGGTAGAACGTCAAC(TGGCGGAACTGC |
| | CGCGCGCGGACACCGCC)₀GGCAGGCCCTGAGCGCCAGTC (SEQ ID |
| | No: 19) |

Notes:
An underlined sequence represents inserted or deleted bases, + represents insertion, and − represents deletion; and a number at a lower right corner of brackets represents a number of repetitions of a sequence in the brackets, for example, 2 indicates two repetitions and 0 indicates deletion.

### (Unique Oligonucleotide Insertion or Deletion Types of the Ultrasonic Treatment Group)

### Example 2: Use of the InDel Molecular Marker in the Detection of the hisD Gene Revertant

(1) In order to intuitively and effectively determine a type of an oligonucleotide insertion or deletion (especially a large-fragment DNA insertion or deletion) of hisD gene in a TA98 revertant, a characteristic primer pair of the InDel molecular marker was designed according to the core mutation region of the hisD gene:

hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

(2) The hisD gene of the TA98 strain and the hisD gene of each of the TA98 revertants obtained in Example 1 were separately subjected to PCR amplification with the primers in step (1), with the PCR procedure being as follows: pre-denaturation at 94° C. for 3 min, denaturation at 94° C. for 20 s, annealing at 55ºC for 30 s, and extension at 72ºC for 1.5 min, with 35 cycles in total; and extension at 72ºC for 10 min.

(3) The PCR product obtained in step (2) was verified by PAGE.

Preparation of a 6% non-denaturing polyacrylamide (PAM) gel: A cleaned and dried vertical electrophoresis tank was arranged, a 1% agar powder gel was melted in a microwave oven and then injected into a gel tank from two sides of a sealing strip, and the edges were sealed. The reagents were thoroughly mixed according to Table 2 and then poured into the gel tank, a 52-gel hole comb was inserted, the excessive gel was removed, and the gel tank was allowed to stand for 20 min, the unsolidified gel was poured out, an appropriate amount of a 1×TBE (Tris-Borate-EDTA) running buffer was added, and the comb was pulled out, and a syringe needle was used to straighten gel holes, and a 200 mL pipette was used to repeatedly flush the gel holes.

TABLE 2
PAGE conditions

| Electrophoresis conditions | Volume |
|---|---|
| 30% PAM | 8 mL |
| 5 × TBE | 8 mL |
| ddH₂O | 23.6 mL |
| 10% Ammonium persulfate (APS) | 400 μL |
| Tetramethylethylene diamine (TEMED) | 40 μL |

The PCR amplification product of hisD gene obtained from the TA98 strain (purchased from MOLTOX™ Molecular Toxicology Inc., USA), the PCR amplification product of hisD gene obtained from the TA98 revertant prepared in Example 1 as a template, and a 6× loading buffer were thoroughly mixed in a ratio of 3:7:2, 1.0 μL of the resulting mixed sample was added to a spotting well with a pipette, and electrophoresis was conducted at a constant voltage of 220 V for 1.5 h.

After the electrophoresis was completed, the gel was taken out and immersed in a 0.1% (w/v) AgNO₃ aqueous solution (50 mL/gel), and shaken at a low speed for 10 min, the solution was poured off, and the gel was washed twice with an equal amount of distilled water, a developing solution (which was prepared by dissolving 8% (w/v) of glucose, 0.7% (w/v) of boric acid, and 1.1% (w/v) of sodium hydroxide in distilled water, and was used at 50 mL/gel) was added, and the resulting mixture was shaken at a low speed until a clear band (for about 10 min to 20 min) appeared, the developing solution was poured off, and the gel was rinsed with tap water 3 times or more, and a gel imaging system was used for photography, and analysis results were shown in the FIGURE.

The FIGURE is an electrophoretogram of all oligonucleotide insertion or deletion types in the core mutant region of hisD in the TA98 revertants. The PCR amplification product of hisD gene of the TA98 strain was adopted as an internal standard, and the meanings of unique oligonucleotide insertion or deletion bands caused by ultrasound were shown in Table 1. It can be seen from the FIGURE that the insertions (+1 to +43 bp) and deletions (−2 to −29 bp) of oligonucleotides were +/− distributed above and below a molecular weight (about 372 bp) of the internal standard (the PCR amplification product of hisD gene of the TA98 strain). Except for the +1 bp insertion and −2 bp deletion, the PAGE electrophoretogram based on the core mutation region can effectively distinguish the band changes caused by insertions or deletions of more than 4 bp, that is, the electrophoretic bands will be distributed above or below the molecular weight (372 bp) of the internal standard.

Therefore, this method can effectively determine a type of an oligonucleotide insertion or deletion of a hisD gene of an S. typhimurium TA98 revertant (an oligonucleotide insertion type was shown above the molecular weight of the internal standard in the electropherogram and an oligonucleotide deletion type was shown below the molecular weight of the internal standard in the electropherogram) to evaluate an effect of ultrasonic mutagenesis, and can also be used to analyze a relationship between a sequence of the hisD gene and a function of a protein encoded thereby.

The above examples are preferred implementations of the present disclosure, but the present disclosure is not limited to the above implementations. Any obvious improvement, substitution, or modification made by those skilled in the art without departing from the essence of the present disclosure should fall within the protection scope of the present disclosure.

What is claimed is:

1. An insertion and deletion (InDel) molecular marker of a hisD gene of an ultrasonically-mutagenized Salmonella typhimurium, wherein the InDel molecular marker is a sequence from an 832^{nd} base to a 915^{th} base in the hisD gene of the ultrasonically-mutagenized Salmonella typhimurium, and has a nucleotide sequence set forth in SEQ ID No: 4.

2. The InDel molecular marker of the hisD gene of the ultrasonically-mutagenized Salmonella typhimurium according to claim 1, wherein the InDel molecular marker is amplified by the following specific molecular marker primers having the following sequences:
hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

3. Primers for detecting the InDel molecular marker of the hisD gene of the ultrasonically-mutagenized Salmonella typhimurium according to claim 1, wherein nucleotide sequences of the primers are as follows:
hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

4. A use of the InDel molecular marker of the hisD gene of the ultrasonically-mutagenized Salmonella typhimurium according to claim 1 in a determination of an insertion or a deletion of a gene reverse mutation.

5. A use of the InDel molecular marker of the hisD gene of the ultrasonically-mutagenized Salmonella typhimurium according to claim 1 in an analysis of a relationship between a sequence of the hisD gene and a function of a protein encoded by the sequence of the hisD gene.

6. A method for detecting an oligonucleotide insertion or a deletion in the hisD gene comprising using the primers according to claim 3 to amplify the InDel molecular marker to obtain an amplification product, wherein the nucleotide sequences of the primers are as follows:
hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

7. The method for detecting the oligonucleotide insertion or the deletion in the hisD gene according to claim 6, wherein with a DNA amplification product of a hisD gene of a histidine-auxotrophic Salmonella typhimurium as a control, a polyacrylamide gel electrophoresis is performed, and if there is a difference in size between bands of the amplification product and the control, a reverse mutation of the oligonucleotide insertion or the deletion occurs.

8. The method for detecting the oligonucleotide insertion or the deletion in the hisD gene according to claim 7, wherein the amplification product is sequenced to analyze a specific oligonucleotide sequence of the oligonucleotide insertion or the deletion.

9. A use of the method according to claim 6 in a detection of a reverse mutation in Salmonella typhimurium mutagenized by a physical field selected from the group consisting of a pulsed electric field and a femtosecond laser.

10. A kit for detecting the InDel molecular marker of the hisD gene of the ultrasonically-mutagenized Salmonella typhimurium according to claim 1, wherein the kit comprises primers for detecting the InDel molecular marker, wherein nucleotide sequences of the primers are as follows:
hisDRC (F):
(SEQ ID No: 20)
5′-GTCAGGTCAGCCAGCGTCT-3′;
and
hisDRC (R):
(SEQ ID NO: 21)
5′-GTAATCGCATCCACCAAATC-3′.

11. The use according to claim 9, wherein with a DNA amplification product of a hisD gene of a histidine-auxotrophic Salmonella typhimurium as a control, a polyacrylamide gel electrophoresis is performed, and if there is a difference in size between bands of the amplification product and the control, a reverse mutation of the oligonucleotide insertion or the deletion occurs.

12. The use according to claim 11, wherein the amplification product is sequenced to analyze a specific oligonucleotide sequence of the oligonucleotide insertion or the deletion.


SEQUENCE LISTING (21 sequences)

SEQ ID NO: 1 - DNA, 24 bp, Artificial Sequence
  Forward primer
atggagtaaagaccatgagcttca

SEQ ID NO: 2 - DNA, 20 bp, Artificial Sequence
  Reverse primer
tgcttgctccttgagggcgt

SEQ ID NO: 3 - DNA, 1305 bp, Salmonella typhimurium
atgagcttcaataccctgattgactggaacagctgtagccctgaacagcagcgtgcgctgctgacgcgtccggcgatttccgcctctgacagtattacccggacggtcagcgatattctggataatgtaaaaacgcgcggtgacgatgccctgcgtgaatacagcgctaaatttgataaaacagaagtgacagcgctacgcgtcacccctgaagagatcgccgccgccggcgcgcgtctgagcgacgaattaaaacaggcgatgaccgctgccgtcaaaaatattgaaacgttccattccgcgcagacgctaccgcctgtagatgtggaaacccagccaggcgtgcgttgccagcaggttacgcgtcccgtctcgtctgtcggtctgtatattcccggcggctcggctccgctcttctcaacggtgctgatgctggcgacgccggcgcgcattgcgggatgccagaaggtggttctgtgctcgccgccgcccatcgctgatgaaatcctctatgcggcgcaactgtgtggcgtgcaggaaatctttaacgtcggcggcgcgcaggcgattgccgctctggccttcggcagcgagtccgtaccgaaagtggataaaatttttggccccggcaacgcctttgtaaccgaagccaaacgtcaggtcagccagcgtctcgacggcgcggctatcgatatgccagccgggccgtctgaagtactggtgatcgcagacagcggcgcaacaccggatttcgtcgcttctgacctgctctcccaggctgagcacggcccggattcccaggtgatcctgctgacgcctgatgctgacattgcccgcaaggtggcggaggcggtagaacgtcaactggcggaactgccgcgcgcggacaccgcccggcaggccctgagcgccagtcgtctgattgtgaccaaagatttagcgcagtgcgtcgccatctctaatcagtatgggccggaacacttaatcatccagacgcgcaatgcgcgcgatttggtggatgcgattaccagcgcaggctcggtatttctcggcgactggtcgccggaatccgccggtgattacgcttccggaaccaaccatgttttaccgacctatggctatactgctacctgttccagccttgggttagcggatttccagaaacggatgaccgttcaggaactgtcgaaagcgggcttttccgctctggcatcaaccattgaaacattggcggcggcagaacgtctgaccgcccataaaaatgccgtgaccctgcgcgtaaacgccctcaaggagcaagcatga

SEQ ID NO: 4 - DNA, 110 bp, Salmonella typhimurium
cctgatgctgacattgcccgcaaggtggcggaggcggtagaacgtcaactggcggaactgccgcgcgcggacaccgcccggcaggccctgagcgccagtcgtctgattgt

SEQ ID NO: 5 - DNA, 84 bp, Salmonella typhimurium
gcccgcaaggtggcggaggcggtagaacgtcaactggcggaactgccgcgcgcggacaccgccggcaggccctgagcgccagtc

SEQ ID NO: 6 - DNA, 85 bp, Salmonella typhimurium
gcccgcaaggtggcggaggcggtagaacgtcaactggcggaactgccgcgcgcggacaccgcccggcaggccctgagcgccagtc

SEQ ID NO: 7 - DNA, 127 bp, Salmonella typhimurium
gcccgcaaggtggcggaggcggtagaacgtcaactggcggaactgccgcgcgcggacaccgccggcaggacgtcaactggcggaactgccgcgcgcggacaccgccggcaggccctgagcgccagtc

SEQ ID NO: 8 - DNA, 118 bp, Salmonella typhimurium
gcccgcaaggtggcggaggcggtagaacgtcaacgcccgcaaggtggcggaggcggtagaacgtcaactggcggaactgccgcgcgcggacaccgccggcaggccctgagcgccagtc

SEQ ID NO: 9 - DNA, 112 bp, Salmonella typhimurium
gcccgcaaggtggcggaggcggtagaacgtcaactggcggaactgccgcgcgcggacaccgctggcggaactgccgcgcgcggacaccgccggcaggccctgagcgccagtc

SEQ ID NO: 10 - DNA, 109 bp, Salmonella typhimurium
gcccgcaaggtggcggaggcggtagaacgtcaactggcggaactgccgcgcgcggacactggcggaactgccgcgcgcggacaccgccggcaggccctgagcgccagtc

SEQ ID NO: 11 - DNA, 106 bp, Salmonella typhimurium
gcccgcaaggtggcggaggcggtagaacgtcaactggcggaactgccgcgcgcggacaccgccactgccgcgcgcggacaccgccggcaggccctgagcgccagtc

SEQ ID NO: 12 - DNA, 103 bp, Salmonella typhimurium
gcccgcaaggtggcggaggcggtagaacgtcaactggcggaactgccgcgaactggcggaactgccgcgcgcggacaccgccggcaggccctgagcgccagtc

SEQ ID NO: 13 - DNA, 100 bp, Salmonella typhimurium
gcccgcaaggtggcggaggcggtagaacgtcaactggcggaactgccgcgcgcggacaccgccgcgcgcggacaccgccggcaggccctgagcgccagtc

SEQ ID NO: 14 - DNA, 94 bp, Salmonella typhimurium
gcccgcaaggtggcggaggcggtagaacgtcaactggcggaaactggcggaactgccgcgcgcggacaccgccggcaggccctgagcgccagtc

SEQ ID NO: 15 - DNA, 94 bp, Salmonella typhimurium
gcccgcaaggtggcggaggcggtagaacgtcaactggcggaacctggcggaactgccgcgcgcggacaccgccggcaggccctgagcgccagtc

SEQ ID NO: 16 - DNA, 94 bp, Salmonella typhimurium
gcccgcaaggtggcggaggcggtagaacgtcaactggcggaactgccgcgcgcggacaccgccggcaggccctggcaggccctgagcgccagtc

SEQ ID NO: 17 - DNA, 91 bp, Salmonella typhimurium
gcccgcaaggtggcggaggcggtagaacgtcaaccgtcaactggcggaactgccgcgcgcggacaccgccggcaggccctgagcgccagtc

SEQ ID NO: 18 - DNA, 91 bp, Salmonella typhimurium
gcccgcaaggtggcggaggcggtagaacgtcaactggcggaacgcggaactgccgcgcgcggacaccgccggcaggccctgagcgccagtc

SEQ ID NO: 19 - DNA, 55 bp, Salmonella typhimurium
gcccgcaaggtggcggaggcggtagaacgtcaacggcaggccctgagcgccagtc

SEQ ID NO: 20 - DNA, 19 bp, Artificial Sequence
  Forward primer
gtcaggtcagccagcgtct

SEQ ID NO: 21 - DNA, 20 bp, Artificial Sequence
  Reverse primer
gtaatcgcatccaccaaatc